(19) United States
(12) Patent Application Publication
(10) Pub. No.: US 2022/0125293 A1
(43) Pub. Date: Apr. 28, 2022

(54) OPTICAL DEVICE AND METHOD

(71) Applicant: THE PROVOST, FELLOWS, FOUNDATION SCHOLARS, AND THE OTHER MEMBERS OF BOARD, OF THE COLLEGE OF THE HOL, Dublin (IE)

(72) Inventors: Gerard BOYLE, Dublin (IE); Mindaugas NORKUS, Dublin (IE)

(73) Assignee: THE PROVOST, FELLOWS, FOUNDATION SCHOLARS, AND THE OTHER MEMBERS OF BOARD, OF THE COLLEGE OF THE HOL, Dublin (IE)

(21) Appl. No.: 17/271,541

(22) PCT Filed: Aug. 29, 2019
(86) PCT No.: PCT/EP2019/073157

§ 371 Date: Feb. 25, 2021

(30) Foreign Application Priority Data
Aug. 29, 2018 (GB) 1814071.5

(51) Int. Cl. A61B 3/00; A61B 5/11; A61B 5/00; A61B 3/113

(52) U.S. Cl. CPC A61B 3/0008; A61B 5/1101; A61B 5/7455; A61B 2560/0431; A61B 3/113; A61B 3/0083; A61B 5/742; A61B 5/7405

(57) ABSTRACT

A device (100) for measuring ocular microtremor (OMT) of a patient's eye, comprises a light source (10) for illuminating a target area of the eye with a light beam. The device also comprises a detector (20) arranged to detect scattered light from the interaction of the light beam with the target area of the eye. The device further comprises a focusing lens (30) arranged to collect the scattered light for the detector, and a port in a wall of the device through which the light beam can exit the device and/or through which the scattered light can enter the device. The device is configured to stabilise and/or support the device on or against a patients head. A method of measuring microtremor of an eye is also provided.

### TECHNICAL FIELD

This invention relates generally to a device for measuring ocular microtremor, in particular based on non-invasive and/or non-contact optical techniques, and a method of measuring the same.

### BACKGROUND TO THE INVENTION

There are a limited number of diagnostic technologies available for quantitative clinical assessment of the status of brain health. Brain imaging technologies, in particular magnetic resonance imaging (MRI) and computed tomography (CT), are extremely powerful investigative tools in neurological conditions. However, by their nature, such imaging systems are large, costly, typically require dedicated installations to which patients are transferred to for scanning, and measurement times are relatively long. The high demand on imaging systems can limit access to the systems and, in any real clinical setting, regular measurements to track brain status are impractical. Non-imaging techniques exist, such as electroencephalography (EEG), but routine/regular use in a clinical setting is typically limited by the complexity of the measurement set-up and interpretation of the results.

There are a limited number of point of care (POC) diagnostic technologies available for quantitative clinical assessment of the status of brain health. It is especially a significant issue in Traumatic Brain Injury (TBI) and concussion. Common clinical practice relies on simple scoring methods, primarily Glasgow Coma Scale (GCS) or similar scales for POC assessment of TBI. Similarly, POC assessment of concussion typically involves some form of cognitive test. Administration of these tests requires a qualified medical professional and is subject to certain limitations, including inter-rater variability, diverged degree of accuracy and lack of clear prognostic capacity.

While several new POC technologies for TBI and concussion have emerged recently primarily employing Blood Biomarkers (BBs) or Electroencephalography (EEG) systems, their administration is not that straightforward or as quick as is desired in a POC setting. BB tests require a blood sample and EEG systems are inherently complex to set-up and interpret. As a result, the widespread uptake of these technologies is limited and slow.

Brain imaging technologies, in particular magnetic resonance imaging (MRI) and computed tomography (CT), are extremely powerful investigative tools in neurological conditions. However, brain imaging is only useful detecting structural damage (such as bleeds) or pathologies in the brain. Most of mild TBI and concussion cases do not show any abnormalities in the brain images post injury. Moreover, by their nature, such imaging systems are large, costly, typically require dedicated installations to which patients are transferred to for scanning, and measurement times are relatively long. The high demand on imaging systems can limit access to the systems and, in any real clinical setting, regular measurements are impractical and, for CT, limited by the radiation dose involved in repeated exposures.

There are three different types of involuntary human eye movement—microsaccades, drift and ocular microtremor (OMT). OMT is the smallest of the involuntary eye movements. It is present in all subjects, even when the eye appears to be at rest. OMT typically has an amplitude in the range 150 nm to 2500 nm and a frequency in the range 20 Hz to 150 Hz. The frequency of OMT is believed to change in a number of neurological conditions such as concussion, coma, multiple sclerosis and Parkinson's disease, and also to correlate with depth of anaesthesia and sedation. OMT is therefore a potentially powerful biomarker that may have a number of practical applications.

To date, most OMT measurements have been carried out using eye-contacting probe techniques, for example utilising piezoelectric probes that must be in physical contact with the eye, as described in U.S. Pat. No. 7,011,410 B2. However, such eye-contacting techniques have a number of drawbacks, such as being invasive, interfering with the eye movements, causing discomfort to the subject, as well as poor reproducibility and accuracy. Non-contact optical techniques of measuring OMT based on laser speckle metrology have been proposed and validated, whereby coherent light reflected from the surface of the eye is imaged and OMT is analysed using image processing methods, e.g. as described by E. Kenny et al., in Journal of Biomedical Optics 18(1), 016010 (2013). However, whilst promising, existing non-contact OMT measurement solutions remain impractical for clinical use.

Aspects and embodiments of the present invention have been devised with the foregoing in mind.

### SUMMARY OF THE INVENTION

According to a first aspect of the invention there is provided a device for measuring ocular microtremor (OMT) of a patient's eye. The device may comprise a light source for illuminating a target area of the eye with a light beam. The device may further comprise a detector arranged to detect scattered light from the interaction of the light beam with the target area of the eye. The device may further comprise a focusing lens arranged to collect the scattered light for the detector. The detector may be positioned in a Fourier plane of the focusing lens. The device may further comprise a port or aperture in a wall of the device through which the light beam can exit the device and/or through which the scattered light can enter the device. The device may further be configured to stabilise and/or support the device on a patient's head.

The detector may be configured to provide one or more output signals representative of the detected scattered light from which one or more OMT properties may be determined.

The device may comprise one or more mounting or support portions or supports. The or each support portion may be configured, in use, to be placed in contact with one or more locations on or around a patient's head or face to support and/or stabilise the device, e.g. the bony parts of the head such as the cheek bone, nose bridge and/or forehead.

Advantageously, the device may be substantially compact and portable, thereby allowing use in a variety of point-of-care (POC) settings, e.g. remote from a fixed clinic or the like (where there are constraints on patient logistics). Further, the device may be configured to perform non-invasive measurements of OMT based on interaction of the light beam with the eye. The term non-invasive is used in this context to mean contactless, such that no direct contact with the eye is required. Advantageously, the device may be configured to measure OMT and provide a rapid assessment of various neurological conditions, e.g. concussion, depth of anaesthesia, and/or brain stem activity.

The device may be configured to perform OMT measurements with a low sensitivity or high tolerance to the tilt of the surface of the target area of the eye and the distance between the device (detector) and the eye, such that the device does not require precise set up. For example, the OMT measurement may be tolerant to relative movement of the device along the illumination axis (i.e. the distance to the eye) on the scale of a few cm without impacting the OMT measurement. This is in part due to the optical arrangement of the lens and detector, whereby scattered light is detected in the Fourier plane of the lens. In this way, the eye is not imaged in the conventional sense, and no precise illumination conditions or adjustment of the lens or other optical elements is required before or during a measurement. The support portions are configured to support and stabilise the device against the patient's head to minimise the movement of the device relative to the patient's head, for example movement substantially in the vertical and/or horizontal plane. The device may comprise one or more supports and/or pads to help counteract unwanted movements in one or more directions.

If OMT is measured in a single direction or plane (e.g. horizontally or vertically) then it may be sufficient to stabilise the device only in the corresponding plane. Alternatively, stabilisation in more than one direction or plane may be utilised. This further improves the reliability and robustness of the measurement, making the device suitable for portable, out-of-clinic and/or handheld applications.

The device may be a handheld device. Alternatively, the device may be mountable to an external support structure such as a wall, frame or movable/articulated arm or mount. As such, in use, the device may be at least partially supported by an external support structure and/or fixed or portable.

The device or housing may be dimensioned and/or configured for an operator to hold and/or grip with one or both hands, e.g. during an OMT measurement. The device may have a dimension (e.g. length and/or width) in the range of substantially 5 cm to 25 cm, for example substantially 10-15 cm long and 5-8 cm wide, or substantially 12 cm long and 5-6 cm wide. Alternatively or additionally, the device may be attachable to an operator's hand by one or more straps or clips.

The device may further comprise a housing or body. The light source, the detector and/or the focusing lens may be located or substantially enclosed within the housing. The housing may comprise the light source, the detector and/or the focusing lens. The port may be located in a wall of the housing. The position of the light source, detector and/or focusing lens may be fixed with respect to the port.

The one or more support portions may be configured to locate on and/or conform to one or more locations on a patient's head or face. At least one of the one or more support portions may be or comprise a projection or extension, extending away from the housing. The one or more support portions may be integrally formed with the housing, or attachable to the housing.

The one or more support portions may further be arranged and configured, when in contact with one or more locations of a patient's head, to position the port at a predetermined location and/or distance from the eye during a measurement. The one or more support portions may further be configured, in use, to position the port within a predetermined volume of space with respect to the eye.

The predetermined distance from the eye may in the range between substantially 1 cm and 5 cm. It may be advantageous to minimise the distance between the port and the eye, for example, to increase the amount of scattered light reaching the detector, and, where the device is a handheld device, to reduce the impact of any hand movements on the position of the port in the vertical plane through the lever action of the support portions.

The one or more support portions may be adjustable and/or moveable relative to the housing and/or vice versa. The orientation, angle and/or position of the or each support portion relative to the housing may be adjustable. The or each supporting portion may be pivotably and/or slidably mountable to the housing. The or each supporting portion may be configured to move in one or more directions relative to the housing. The directions may be linear (e.g. X, Y and/or Z), or non-linear (e.g. rotation). The or each support portion may be manually adjustable and/or moveable. Alternatively or additionally, adjustment and/or movement of the or each support portion may be electrically controlled, e.g. the device may be configured to adjust and/or move the support portion(s) relative to the housing (or vice versa) in response to an operator input. For example, the device may comprise one or more motors or actuators (e.g. piezo, servo and/or stepper motors) arranged and/or configured to adjust and/or move the support portion(s) relative to the housing (or vice versa) in response to an operator input.

It will be appreciated that the movement/adjustment is relative, such that when either one of the support portion(s) and housing are held in a fixed position, the other will move relative to it. In use, the support portion(s) are placed in contact with the patient's head/face, such that the housing moves relative to the support portion(s). However, it will be appreciated that movement/adjustment of the device may be performed before, during and/or after a measurement.

Advantageously, relative movement between the or each support portion and the housing may enable the position of the port (and optical elements within the housing) relative to the eye and/or the one or more location on patient's head/face to be controlled. This may be particular advantageous in locating the target area of the eye, where the dimensions of each patient's may be different. In addition, this may advantageously enable the target area of the eye to be located by adjusting or aligning the device without having to reposition the device repeatedly on the patient's head/face. Relative movement between the or each support portion and the housing may further allow adjustment of the target area of the eye illuminated with the light beam. Automated/semi-automated and/or electrically controlled movement may be advantageous in locating/adjusting the target area in a controlled manner, and/or measuring a number of target areas on the patient's eye (e.g. in a measurement sequence) in a controlled manner. This function may improve the reliability of the measurement, for example, it be used to obtain a measurement data set from which anomalous data can be identified and discarded.

Automated/semi-automated movement may be further advantageous in locating or aligning the device to the target area of the eye during the measurement set-up phase. For example, the device may be configured to scan the light beam over an area of the eye and locate the target area based on the detector output. For example, the device may be configured to scan an area of the eye to produce an intensity map of detected light scattered from the eye. Contrast in the intensity map may reveal gross features of the eye (e.g. eye lids, sclera and pupil) from which the target area may be selected, and the device then aligned to.

Relative movement between the support portion(s) and the housing may be course and/or fine. Course movement may provide a longer range of movement but with lower positional resolution than fine movement. Course movement may have a positional resolution down to substantially 0.5 mm, 0.2 mm or 0.1 mm. Fine movement may have a position accuracy of less than substantially 0.5 mm, 0.2 mm, or 1 mm. For example, manual movement provide for course movement, and electrically controlled movement provide fine movement. Alternatively, manual movement or electrically controlled movement may provide both course and fine movement.

The or each support portion may be attachable to the housing via a translation stage. The translation stage may be or comprise a manual and/or electrically controlled translation stage (e.g. comprising piezo actuators, magnetic drives, linear motors, encoders, or motors, or other translations stage technologies known in the art). The translation stage may provide course and/or fine movement. The housing may comprise the translation stage, or the support portion(s) may comprise the translation stage. The translation stage may be configured to translate the or each support portion relative to the housing in one or more directions (e.g. orthogonal X, Y and Z directions, rotation and/or tilt). The translation stage may be or comprise a multi-axis translation stage. The one or more support portions may be attachable to the housing and/or the translation stage by one or more additional couplings or mounts, e.g. fixed, or pivotable couplings.

The one or more support portions may be removable from the housing. This may permit exchange for different sized and/or shaped support portions. For example, different support portions may be configured to contact and/or conform to different locations on a patient's head or face (e.g. the cheek bone, forehead, or nose bridge etc.) and thus positon the port at different positions/locations relative to the point of contact of the support portion with the patient's head/face.

The device may be modular, comprising one or more sub-units or modules. The housing may be or comprise a measurement module. The light source, the detector and/or the focusing lens may be located or substantially enclosed within the measurement module. The device may comprise a support module. The support module may be or comprise the one or more support portions. The support module may be adjustable and/or moveable relative to the measurement module or vice versa.

At least one of the one or more support portions may be arranged to provide, in use, a pivot point (e.g. when in contact with a location on a patient's head or face) about which the position (in the vertical plane and/or the horizontal plane) of the device relative to the eye can be adjusted. The pivot point may allow an operator to adjust the distance and/or location of the port with respect to the eye before, during and/or after a measurement. The pivot point may further allow an operator to adjust the target area of the eye illuminated with the light beam.

The device may further comprise one or more visual targets for the patient to look at during a measurement. The target(s) may be a physical object mounted to the housing or part of the housing, or the target may be or comprise a second light source mounted to the housing and configured to emit visible light (or a combination thereof). This may be used to guide the patient's eye to a predetermined orientation/position relative to the light beam. The device may be configured to move the visual target(s) relative to the housing. For example, the visual target may be mounted to the housing via a manual and/or electrically controlled translation stage. In this way, for a fixed position of the port (and light beam) relative to the patient's head/face, the illuminated target area of the eye can be adjusted as the patient's eye follows the visual target(s). As such, this may also be used as an additional/alternative approach to adjusting and/or locating the target area of the eye.

The support portion(s) may be configured to minimise movement of the support portion(s) relative to the patient's head during a measurement. The support portion(s) are intended to be a point of stability. The one or more support portions may be configured to firmly or rigidly couple to the patient's head such that, in use, the device may follow any movements of the patient's head. In this way, the one or more support portions may provide effective passive stabilisation to the device.

The or each support portion may comprise an interface portion configured to be placed in firm or fixed contact with the patient's head. The interface portion may be positioned at a distal end of the or each support portion. The one or more support portions and/or interface portion may be formed of or comprise a substantially hard material. The or each interface portion may be or comprise a pad. The one or more support portions and/or the interface portion may be ergonomically shaped to conform to one or more locations on a patient's head or face. Optionally or preferably, the one or more support portions and/or the interface portion may be ergonomically shaped to conform to one or more bony areas of the patient's head or face, such as the forehead, cheek, eye socket or nose bridge.

The or each interface portion may be attachable to the respective support portion. The or each interface portion may be firmly coupled to the respective support member. Alternatively, the or each interface portion may be coupled to the respective support portion via an anti-vibration coupling configured to dampen vibrations transferred from the patient's head to the device (or vice versa) and/or the operator to the patient's head. The anti-vibration coupling may comprise an anti-vibration material. The anti-vibration material may be or comprise a substantially soft and/or resilient material.

The device may further comprise a handle for holding and/or manipulating the device by an operator. Optionally or preferably, the handle may comprise an ergonomic grip. The handle may be joined or coupled to the housing or the support portion(s) or integrally formed therewith. The handle may be or comprise an anti-vibration handle. The handle may comprise an anti-vibration material configured to dampen vibrations. For example, the grip may be or comprise the anti-vibration material. Alternatively, the coupling between the handle and the housing or the support portion(s) may be or comprise an anti-vibration coupling. The anti-vibration coupling may comprise the anti-vibration material. The anti-vibration material may be or comprise a substantially soft and/or resilient material.

The anti-vibration material may be or comprise a rubber like material, such as Nitrile, neoprene, polyurethane and/or any other vibration damping material known in the art. Alternatively or additionally, the anti-vibration material may be or comprise a resilient member such as a spring or the like.

Advantageously, the handle may be configured to substantially decouple or isolate vibrations from an operator's hand(s) such that transfer of hand movements and/or vibrations to the device is substantially reduced.

The handle may be or comprise a portion e.g. an elongated portion dimensioned and/or shaped to be held in an operator's hand. Alternatively, an aperture may be provided within the housing to accommodate an operator's hand or at least some part thereof.

Alternatively or additionally, the device may further comprise an active stabilisation mechanism, such as an anti-shake mechanism, to reduce the impact of operator hand and/or patient head movement on the OMT measurement.

The handle may be or comprise part of the housing. The handle may be integrally formed with the housing, or attachable (e.g. fixedly or pivotably) to the housing. The handle may be firmly coupled to the housing. Where the housing is configured to move relative to the support portion(s) (or vice versa), the handle may be configured to move with the housing (e.g. where it is part of the housing or attachable to the housing), such that both the housing and the handle move relative to the support portion(s).

Alternatively, the handle may be or comprise part of the support portion(s). The handle may be integrally formed with the support portion(s), or attachable (e.g. fixedly or pivotably) to the support portion(s). The handle may be firmly coupled to the support portion(s) or softly coupled via an anti-vibration coupling. As such, where the housing is configured to move relative to the support portion(s) (or vice versa), the handle may be configured to be stationary with respective to the support portion(s), such that the housing moves relative to the support portion(s) and the handle.

Alternatively, each of the housing and the support portion(s) may be attachable (e.g. fixedly or pivotably) to the handle. Alternatively or additionally, the housing and/or support portions may be adjustable and/or moveable with respect to the handle (or vice versa).

The handle may be a handle module of the device. The handle module may be attachable to, and/or adjustable or moveable with respect to, the measurement module and/or support module. As such, each of the support module, the handle module and the measurement module may be configured to move independently with respect to each other.

The orientation, angle and/or position of the handle relative to the housing and/or support portion(s) may be adjustable. The handle may be pivotably and/or slidably mountable to the housing and/or the support portion(s). The housing and/or support portion(s) may be configured to move in one or more directions relative to the handle. The directions may be linear (e.g. X, Y and/or Z), or non-linear (e.g. rotation). The housing and/or support portion(s) may be manually adjustable and/or moveable relative to the handle. Alternatively or additionally, adjustment and/or movement of the support portion(s) and/or housing relative to the handle may be electrically controlled, e.g. the device may be configured to adjust and/or move the support portion(s) and/or the housing relative to the handle in response to an operator input. The device may comprise one or more motors or actuators (e.g. piezo, servo and/or stepper motors) arranged and configured to adjust and/or move the support portion(s) and/or the housing relative to the handle in response to an operator input.

The handle may be attachable to the support portion(s) and/or the housing via a respective translation stage. The or each translation stage may be or comprise a manual and/or electrically controlled translation stage. The or each translation stage may provide course and/or fine movement. The handle may comprise the or each translation stage, or the support portion(s) and/or the housing may comprise the respective translation stage. The or each translation stage may be configured to translate the respective support portion(s) or housing relative to the handle in one or more directions (e.g. orthogonal X, Y and Z directions, rotation and/or tilt). The or each translation stage may be or comprise a multi-axis translation stage. The support portion(s) and/or the housing may be attachable to the respective translation stage by one or more additional couplings or mounts, e.g. fixed, or pivotable couplings.

The housing or measurement module may be at least partially received within the handle or handle module.

The detector may be arranged and configured, in use, to image the scattered light in the Fourier plane of the focusing lens. The detector may be arranged and configured, in use, to image an interference pattern formed in the Fourier plane of the focusing lens. The interference pattern may be a speckle pattern.

The detector may be or comprise an imaging sensor. The detector/imaging sensor may be configured to capture and output a rapid succession of images or image frames of the scattered light (e.g. the interference pattern) over a period of time (i.e. the acquisition time). Optionally or preferably, the imaging sensor may have a frame rate of at least 500 Hz. The imaging sensor may be configured to capture one or more one-dimensional images and/or two-dimensional images. The imaging sensor may be or comprise a one dimensional or two-dimensional array of individual sensing elements, or an array of two-dimensional image sensors. A one dimensional image may be used to measure OMT in one particular direction (e.g. X, Y, or vertical or horizontal directions). The output of the detector/imaging sensor may be a video comprising a number of images or image frames recorded over a period of time. The acquisition time may be less than substantially 10 seconds. The acquisition time may be in the range of substantially 2 to 10 seconds, 3 to 6 seconds or 4 to 5 seconds. The video may have a length of 3 seconds and/or comprise 1500 images. The imaging sensor may be or comprise a high speed digital camera.

The device may be configured to perform OMT measurements based on an output of the detector. The device may be configured to measure the horizontal component of the OMT and/or the vertical component of the OMT. The device may be configured to perform OMT measurements based on optical interferometry and, optionally or preferably, speckle metrology.

The device may further comprise a processor configured to perform the OMT measurement based on an output of the detector. The processor may be or comprise an integrated circuit or a micro-processor configured to process the output of the detector. The processor may be configurable, e.g. a field programmable gate array. Alternatively, the device may be connectable to a processor configured to perform the OMT measurement based on an output of the detector (e.g. a processor on a separate computing device). The processor may be configured to determine a microtremor amplitude and/or frequency based on the output of the detector. The processor may be configured to receive a detector output signal. The detector output signal may be or comprise the detector output. The detector output signal may be or comprise a stream of image data to the processor, such as a stream or series of image frames or a video stream. The processor may be configured to record the output of the detector over a period of time. The detector output signal may be or comprise a series of image frames captured over a period of time at a frame rate. Each image frame may comprise image data. The acquisition time or period of time may be in the range of substantially 2 to 10 seconds, 3 to 6 seconds or 4 to 5 seconds. The processor may be configured to determine or execute a software algorithm for determining the microtremor amplitude and/or frequency from the recorded detector output.

The OMT measurement may be based on optical interferometry, optionally or preferably, speckle metrology. Light received at the detector may form an interference or speckle pattern, which can be detected and/or captured by the detector. Movement of the eye causes changes and/or displacements in the interference pattern over time. The speckle pattern or interference pattern may be an image. The processor may be configured and/or programmed to process and/or analyse the changes in the interference or interference pattern to extract an OMT signal. The OMT signal is a time varying signal which is an estimate of the eye movement caused by OMT. The OMT signal is further processed to extract the dominant frequency present in the OMT signal. Measurement of an OMT frequency below a certain threshold level may be indicative of an abnormality in the patient's brain health, and in the context of the device's use, may be indicative of a brain injury. The analysis may look for unexpected patterns or changes over time.

The processor or the algorithm executed by the processor may be configured to cross-correlate images received from the detector to produce the OMT signal representative of eye movement over time. The OMT signal may be substantially oscillatory and comprise a plurality of frequency components. The algorithm may include appropriate filtering steps to extract the frequency components of the detector output signal in the frequency band of interest, or attenuate the frequency components outside the frequency band of interest. The frequency band of interest for OMT may be in the range of substantially 20 Hz to 150 Hz. Frequencies outside of the band of interest may represent microsaccades and/or eye drift. The algorithm may include steps to estimate signal frequency content by determining peak spectral components through Fourier analysis or by determining signal peaks occurring per second.

The processor or software algorithm may be configured to cross-correlate each image, starting from the second image in the stream of image data, with the previous image in the stream of image data. The peak of the cross-correlation result may represent the speckle displacement between the two cross-correlated images, e.g. at a pixel resolution. The OMT signal may comprise the location of the cross-correlation peak over time. The processor may be configured to filter the OMT signal (e.g. by applying a band-pass filter), to extract the frequency components of the detector output signal in the frequency band of interest, or attenuate the frequency components outside the frequency band of interest.

The processor may be configured to: receive an output signal from the detector, the detector output signal comprising a stream of image data comprising a series of image frames captured over a period of time at a frame rate, and reject or delete image frames from the image data having one or more image frame quality metrics falling outside one or more predefined threshold values. The processor may be configured to determine, for one or more image frames of the series of images, one or more image frame quality metrics based on respective image frame data. The processor may be configured to compare, for each of the one or more image frames, the one or more image frame quality metrics to one or more predefined threshold values. The one or more image frame quality metrics may include a total integrated signal across a respective image frame, and/or a height and/or width of an auto correlation peak obtained from a respective image frame.

Alternatively or additionally, the processor may be configured to reject or delete pairs of image frames from the image data having one or more inter-frame correlation quality metrics falling outside the one or more predefined threshold values. The one or more inter-frame correlation quality metrics may include one or more signal-to-noise parameters extracted from the cross-correlation of a respective pair of image frames.

The processor may be configured to determine or extract an OMT signal from the remaining image data representative of eye movement over time. The OMT signal may be determined or extracted from the cross-correlation of adjacent pairs of images in the remaining image data. Cross-correlation of a pair of image frames may produce cross-correlation data or a cross-correlation matrix for the pair of image frames. The OMT signal may be determined or extracted from the cross-correlation data.

Alternatively or additionally, the processor may be configured to reject or delete image frames from the image data having an inter-frame velocity exceeding a predefined threshold value. The inter-frame velocity may be based on the change in cross-correlation peak position between successive pairs of cross-correlated image frames.

The device may further comprise a control circuit or microcontroller configured to operate the light source and detector. The control circuit may be in communication with the light source, detector and/or processor. The control circuit may comprise the processor. The control circuit may further be configured to control movement/adjustment of the support portion(s), housing and/or handle. The control circuit may be configured to control the light source, detector, processor, the or each motor or electrically controlled translation stage in response to an operator input.

The device may further comprise one or more operator inputs for operating the device. One or more of the operator inputs may be arranged and configured, in use, to be operable by an operator's hand (e.g. using a finger and/or a thumb) whilst holding and/or gripping the device with said hand. The one or more operator input may be arranged on or in the housing and/or handle, or the measurement module and/or handle module.

The one or more operator inputs may be connected to or in communication with the control circuit and/or the processor The one or more inputs may include, but are not limited to: switches, buttons, touch pads, joysticks, keypads, trackpads, direction pad, multi-direction buttons, and/or microphones. The one or more inputs may be configured to perform one or more of the functions: on/off; initiate OMT measurement; clear or interrupt measurement; terminate measurement e.g. before a measurement period has expired; move or adjust the position of the support portion(s), housing and/or handle.

The device may further comprise one or more output elements configured to provide one or more output signals to an operator. The one or more output signals may be or comprise an audio, a visual and/or a haptic signal. For example, the one or more output elements may be or comprise a sound emitting device, a light emitting device, and/or a haptic device such as a vibrator. The one or more output elements may be configured to indicate one or more types of information to an operator. For example, the one or more types of information may be or comprise a measurement result (e.g. OMT amplitude and/or frequency), a measurement start, a measurement stop, an OMT trace, and/or a measurement feedback.

At least one of the output elements may be or comprise a display screen. The display screen may be configured to display measurement data including one or more of: a measurement status, a measurement result, a measurement start, a measurement stop, an OMT amplitude, an OMT frequency, and/or an OMT trace.

The processor may be in communication with the one or more output elements. The processor may be configured to provide one or more feedback signals to the one or more output elements for providing to the operator, based on the one or more image frame quality metrics and/or inter-frame correlation quality metrics. The one or more feedback signals may provide device alignment and/or stability feedback to the operator.

The lens may be a fixed focal length lens. For example, the lens may be or comprise a convex or plano-convex lens. The focal length may be less than 10 cm. The focal length may be in the range of substantially 2 cm to 10 cm, 3 cm to 10 cm, 4 cm to 10 cm, 5 cm to 10 cm, 5 cm to 9 cm, or 5 cm to 8 cm.

The detector may be positioned at a distance substantially equal to the focal length from the lens. A plurality of lenses may instead be used instead of a single lens to focus scattered light at the detector position

The device may comprise an illumination optical path by which the light beam reaches the port from the light source, and a detection optical path by which the scattered light reaches the detector from the port.

The device may further comprise a beam splitter. The beam splitter may be arranged in the illumination optical path to direct the light beam towards the port and/or arranged in the detection optical path to direct the scattered light to the detector.

The device may further comprise a mirror. The mirror may be arranged in the illumination optical path to direct the light beam towards the port. The mirror and or the beam splitter may be housed within the housing or measurement module.

A configuration with a mirror allows the light source to be arranged in a variety of positions within the housing. It may be advantageous for the operator to see the eye during alignment, and this arrangement may allow the light source to be positioned such the operator has a less obstructed view of the patient's eye. The device may be configured such that the operator can view the patient's eye through the beam splitter. This may further aid alignment of the device to the target area on the eye.

The mirror may be or comprise a moveable mirror to adjust the position of the light beam. The movable mirror may be manually operated or electrically controlled (e.g. using motorised mirror mounts, piezo actuators, servos or any other moveable mirror technologies known in the art). The moveable mirror may be used in combination with, or instead of, a moveable housing, support and/or handle module to aid in aligning the light beam to the target area of the eye. Where the mirror is electrically controlled, the device may be configured to move the mirror in response to one or more inputs from the operator. The device may be configured to perform an auto-locate or auto-align function in response to an operator input, whereby the mirror is moved in such a manner to scan the light beam across the eye. In an auto-locate function, the device may further be configured to move the mirror in response to a detector output signal to locate the target area whilst maintaining the scattered light in the field of view of the detector (i.e. to maintain a sufficient detector signal).

The light source may be configured to emit coherent light. The light source may be configured to emit substantially monochromatic light. The light source may be or comprise a laser. Optionally or preferably, the laser may emit light at one or more wavelengths in the visible to near-infrared wavelength range (e.g. in the range of about 550 nm to about 850 nm, or 633 nm to about 785 nm). Optionally, the device may comprise one or more light sources, each light source configured to emit light at a different wavelength. For example, one light source may be configured to emit light in the visible wavelength range (e.g. 400-700 nm), and another light source may be configured to emit light in the near-infrared wavelength range (e.g. 700 to 900 nm). Both light sources may be arranged to emit a light beam along the same or parallel illumination paths to the eye, such that both light beams illuminate substantially the same target area of the eye. The visible light source may be used for alignment purposes (i.e. so the operator can see the light beam), and the near infra-red light source may be used for OMT measurement, once the device or light beam is aligned to the target area.

The device may be configured to provide a light beam at the port with an optical power less than substantially 150 μW (e.g. an eye safe level at the wavelength of the light beam—determined by laser safety standards). Optionally or preferably, the device may comprise one or more attenuating optical elements arranged in the illumination optical path to reduce the power of the light beam exiting the device to an eye safe power at the wavelength of the light beam. For example, a light source may emit light at substantially 1 mW power, which must be attenuated to an eye safe level.

The device may further comprise one or more optical band-pass filters. The optical band pass filter(s) may be arranged in the detection and/or illumination optical paths. The optical band-pass filter may be configured to transmit the scattered light and attenuate ambient light reaching the detector.

The device may further comprise a shutter. The shutter may be arranged in the illumination path to selectively block the light beam, e.g. to prevent the light beam from exiting the device through the port. The shutter may be positioned in the illumination optical path in front of or behind the port. The shutter may be manually or automatically operated (e.g. by the control circuit in response to an operator input via the one or more operator input elements).

The device may be battery powered. The device may comprise a battery. The battery may be rechargeable. The battery may be connectable to the control circuit to provide power to the control circuit. Alternatively, the device may be mains powered, e.g. via a power cord. Where the device is mains powered, the control circuit may receive mains power via a power cord.

The device may further be configured to measure movement of the eye relative to movement of the patient's head. In this way, the impact of movement of the patient's head and/or the operator's hand(s) on the OMT measurement can be eliminated or substantially reduced. For example, the device may be further configured to measure displacement of a reference target at or on the patient's head. The reference target may be configured to contact a location of the patient's head such that any movement of the patient's head is transferred to the reference target. The reference target may be substantially decoupled from the device such that it can move independent from the device. The device may be configured to measure the displacement of the reference target with respect to the housing and the movement of the eye with respect to the housing. From these two measurements, the device may be configured to determine the difference in movement between the eye and the reference target. The reference target may be part of the device. The reference target may be or comprise part of the support portion(s). For example, where the support portion(s) comprise an interface portion that is coupled to the support portion(s) via an anti-vibration coupling, the reference target may or comprise part of the interface portion.

The device may comprise a first sub-system for measuring angular movements or displacements of said eye relative to the device based on an output signal of the detector, and a second sub-system for measuring angular movements or displacements of said head relative to the device based on an output signal of a second detector. The first sub-system may comprise the detector and the focusing lens. The first sub-system may be configured to provide a first signal representing angular movements or displacements of said eye relative to the device based on an output signal of the detector. The second sub-system may comprise a second detector arranged to detect scattered light from the interaction of the light beam with a reference target at or on the patient's head, and a second focusing lens arranged to collect the scattered light for the second detector. The second sub-system may be configured to provide a second signal representing angular movements or displacements of said head relative to the device based on an output signal of the second detector. The device is configured to provide an OMT signal representing angular movements or displacements of said eye relative to said head based on the first and second measurement signals.

The processor may be configured to determine the first and second signals based on the output signals of the detector and the second detector. The processor may be configured to determine the OMT signal based on the first and second signals by combining the first and second signals.

It will be appreciated that the device acts as an interferometer using an interference (speckle) pattern generated by the interference of scattered light with itself after interacting with the target area of the eye. Alternatively, the displacement of the eye relative to the device may be measured using an interference pattern generated by the interference of the scattered light with a reference light beam that has not interacted with the target area of the eye, in a Michelson interferometer type arrangement. Such an arrangement would require an additional mirror arranged on the other side of the beam splitter to the incident light beam (from the light source), e.g. in place of a beam dump.

The device may further be configured perform a retinal scan to identify a patient, using known techniques. OMT measurement data stored by the device or transmitted via the communication unit may include retina scan data as biometric identifier. For example, the detector may further be configured to capture images of the patient's retina. Alternatively, the device may comprise a separate detector arranged and configured for obtaining retinal images. The processor may further be configured to execute a retina recognition algorithm using stored retina scan data, e.g. stored on the device or accessible via the communication unit. This may enable the device to identify a patient and associate OMT measurement with a patient.

Although the features of the device have been described in terms of measuring OMT of an eye, it will be appreciated that the device may equally be suitable for and used to measure other eye movements (e.g. occurring in different frequency ranges) and microtremor and/or small displacements of inanimate objects.

According to a second aspect of the invention, there is provided a method of measuring microtremor of an eye. The method may comprise providing a device according to the first aspect. The method may comprise supporting and/or stabilising the device on or against the patient's head. Supporting and/or stabilising the device may comprise positioning the device such that a support portion of the device is in contact with one or more locations on a patient's head or face. The method may further comprise aligning the device to a target area of the eye. The method may further comprise illuminating the target area of the eye with a light beam emitted from the device and detecting scattered light from the interaction of the light beam with the target area of the eye at a detector of the device. The method may further comprise determining or measuring one or more microtremor properties based on an output signal of the detector. The one or more microtremor properties may be or comprise one or more amplitude and/or frequency components of the output signal. The method may further comprise providing and/or displaying at least one of the one or more microtremor properties to an operator.

Where the device comprises one or more support portions, the method may further comprise the steps of: placing the one or more support portions in contact with one or more locations on a patient's head or face; and adjusting the position of the light source, detector and focusing lens to align the device to the target area of the eye.

Aligning the device may comprise aligning the light beam with the target area of the eye. Aligning the device may further comprise adjusting and/or moving the housing and/or support portions. Aligning the device may comprise adjusting and/or moving the housing relative to the support portion(s). Adjusting and/or moving the housing and/or support portions may comprise adjusting and/or moving the housing and/or support portions while the device is supported by on or against the patient's head.

The method may comprise steps of performing any of the functions of the device of the first aspect.

According to a third aspect of the invention, there is provided a mobile computing device configured to perform the method of the second aspect. The mobile computing device may be or comprise a tablet, smart phone, or laptop.

References to “light” throughout are intended to refer to light at least in the visible and/or near-infrared parts of the spectrum. Radiation of other wavelengths may also be utilised in other embodiments.

Features which are described in the context of separate aspects and/or embodiments of the invention may be used together and/or be interchangeable. Similarly, where features are, for brevity, described in the context of a single embodiment, these may also be provided separately or in any suitable sub-combination. Features described in connection with the device may have corresponding features definable with respect to the method(s), and vice versa, and these embodiments are specifically envisaged.

### BRIEF DESCRIPTION OF DRAWINGS

In order that the invention can be well understood, embodiments will now be discussed by way of example only with reference to the accompanying drawings, in which:

FIG. 1 shows a schematic diagram of the optical set up of a device according to an embodiment of the invention;

FIG. 2 shows an illustration of a device according to an embodiment of the invention;

FIG. 3 shows a schematic diagram of a device according to an embodiment of the invention;

FIG. 4 shows another diagram of a device according to an embodiment of the invention;

FIG. 5 depicts a method of using the device of FIGS. 1 to 4 according to an embodiment of the invention;

FIG. 6 shows a device configuration according to another embodiment of the invention;

FIGS. 7a and 7b show ray diagrams for compensating for angular movements of a device; and

FIGS. 8a and 8b show alternative optical set ups of a device for compensating for angular movements of the device.

### DETAILED DESCRIPTION

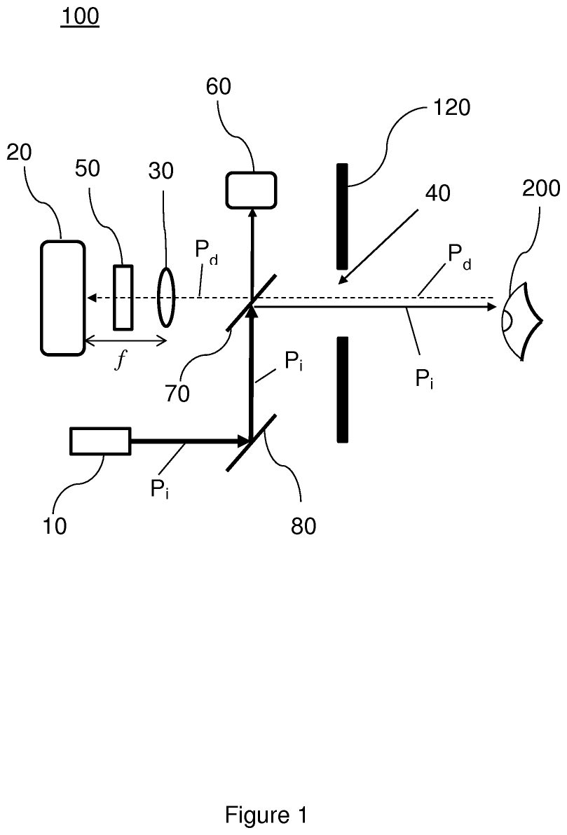

FIG. 1 shows a schematic diagram of a device 100 according to an embodiment of the invention. The device 100 is an optical measurement device configured for measuring small displacements of a target object, in particular, ocular microtremor (OMT) of an eye. The device is configured to illuminate a target area of an eye with a light beam and detect scattered light from the eye. The detected scattered light forms an interference pattern that changes or moves with time as the eye moves. The interference pattern can be imaged, recorded and processed to determine one or more OMT properties (e.g. amplitude and/or frequency) of the eye.

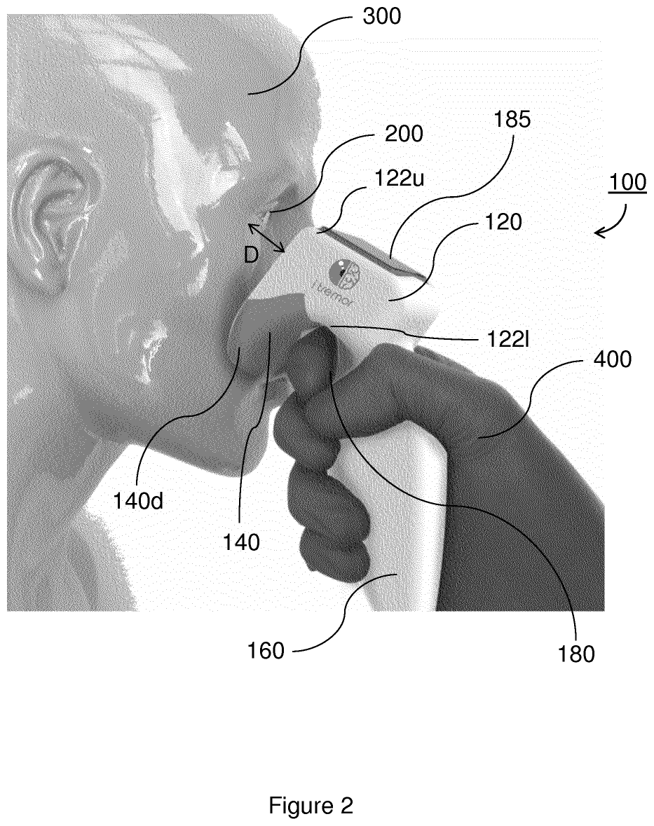

The device 100 may be configured and dimensioned to be a handheld device 100, such that it can be held and/or operated using a single hand 400, e.g. as shown in FIG. 2. Alternatively, the device 100 may be configured and dimensioned to be at least partially support by an external support structure such as a wall or frame, such that it is not entirely hand held.

The device 100 comprises a plurality of optical elements. The device 100 comprises a light source 10 for illuminating a target area of an eye 200 with a light beam. The target area may be the eye sclera (white of the eye). The device 100 further comprises a detector 20 arranged to detect scattered light from the interaction of the light beam with the target area of the eye 200. For example, the scattered light reaching the detector 20 may be or comprise a fraction of the light beam that is reflected from the target area.

The device 100 comprises a focusing lens 30 arranged to collect the scattered light for the detector 20. The focusing lens 30 may be arranged in front of the detector 20 and positioned relative to the detector 20, such that the detector 20 is positioned in a Fourier plane of the focusing lens 30. To achieve this, the lens 30 may be positioned at a predefined distance from the detector 20 equal to the focal length f of the lens 30.

An advantage of the Fourier plane technique is that the OMT measurement is relatively insensitive to the tilt or shape of the surface of the target area and the distance between the target surface and the detector. Further, the angular displacement of the eye is dependent only on the linear speckle image displacement and the focal length of the lens. As such, the optical setup of the device is less stringent than that of systems detecting in the imaging plane where the speckle images are dependent on a number of variables including the wavelength of light and the detector-target distance.

The focal length f provides a magnifying effect to the motion of the eye. As such, for a given detector, the longer the focal length the better the resolution of device to (rotational) eye motion. Preferably, the focal length f of the lens 30 is less than substantially 10 cm to allow the device 100 to be substantially compact and portable whilst providing adequate resolution (preferably in the range substantially 2-5 μrad or lower).

The light source 10, the detector 20 and the focusing lens 30 are housed in a housing 120 of the device 100. The housing 120 may substantially enclose the optical elements of the device 100, such as the light source 10, the detector 20 and the focusing lens 30. The housing 120 may comprise a port, opening or aperture 40 in a wall of the housing 120, as shown in FIG. 1. The aperture 40 may allow the light beam to exit the housing 120, to illuminate the target area of the eye 200. The aperture 40 further allows scattered light from the target area of the eye 200 to enter the housing 120 and reach the detector 20.

The device 100 comprises an illumination optical path, by which the light beam reaches the aperture 40 and/or the target area of the eye 200 from the light source 10, and a detection optical path, by which the scattered light reaches the detector 20. In FIG. 1, the illumination optical path is indicated by solid arrows labelled Pᵢ and the detection optical path is indicated by dashed arrows labelled P_{d}. Although FIG. 1 shows the detection and illumination optical paths (Pᵢ, P_{d}) passing through the same aperture 40, in an alternative arrangement (not shown), the detection and illumination optical paths (Pᵢ, P_{d}) may each pass through a separate aperture 40. The focusing lens 30 is positioned in the detection optical path P_{d}.

The or each aperture 40 may be or comprise an optical window (not shown). The optical window 40 may be optically clear and/or substantially transparent in the wavelength range of interest, e.g. the wavelength range of light emitted by the light source 10 and/or the scattered light from the eye 200. In this way, the housing 120 may fully enclose and/or seal the optical elements within the housing 120 to protect the optical elements from the environment (e.g. from dust or other particulates). The or each window 40 may be or comprise an optical band pass filter configured to transmit the light beam and/or the scattered light in the wavelength range of interest and attenuate ambient light.

Additionally or alternatively, the device 100 may comprise an optical bandpass filter 50 arranged in the detection optical path P_{d} front of the detector 20, as shown in FIG. 1. The optical bandpass filter 50 may be configured to transmit the scattered light in the wavelength range of interest and attenuate ambient light from reaching the detector 20.

The device 100 may further comprise a beam splitter 70 arranged in the illumination optical path Pᵢ to direct the light beam towards the aperture 40. The beam splitter 70 is configured to exhibit partial reflectance R (i.e. R<100%) and partial transmittance T (i.e. T<100%) in the wavelength range of interest. As such, the optical intensity of reflected and transmitted light is less than that of the light incident upon the beam splitter 70. This effect is illustrated in FIG. 1 by the reduced widths of the solid arrows after the beam splitter 70.

The beam splitter 70 may also be arranged in the detection optical path P_{d} to direct the scattered light towards the detector 20, as shown in FIG. 1. In the example arrangement shown, the light beam from the light source 10 is partially reflected from the beam splitter 70 along the illumination optical path Pᵢ to the aperture 40/eye 200, and the scattered light from the target area of the eye 200 travels back towards, and is partially transmitted through, the beam splitter 70 along the detection optical path P_{d} to the detector 20. It will be appreciated that the beam splitter 70 may have any suitable splitting ratio R:T for performing the measurement. For example, the suitable splitting ratio R:T may be chosen to maximise the amount of scattered light reaching the detector. The splitting ratio R:T may depend upon the intensity of the light beam incident on the beam splitter 70, the intensity of scattered light form the eye 200, and/or the sensitivity of the detector 20. Since the intensity of scattered light will typically be less than the intensity of the light beam illuminating the eye 200, in an arrangement such as that shown in FIG. 1, the beam splitter 70 preferably has a reflectance R<50% in order to transmit a larger proportion of scattered light from the eye. In an embodiment, the splitting ratio is approximately 10:90 (reflection % to transmission %) in the wavelength range of interest. The relatively high transmittance may increase the amount of scattered light transmitted to the detector 20 and hence improve the signal to noise ratio of the measurement.

Where a beam splitter 70 is used in the optical set-up, the device 100 may further comprise a beam stopper 60 or beam dump 60 arranged to block and/or terminate the portion of the light beam transmitted through the beam splitter 70 (which is not used to illuminate the eye 200). Preferably, the beam dump 60 may be substantially non-reflective or absorbing to help prevent any residual light beam from reaching the detector 20. The beam splitter 70 and/or the beam dump 60 may be located in the housing 120.

Although the illumination optical path Pᵢ and the detection optical path P_{d} at the beam splitter 70 are substantially orthogonal in the arrangement of FIG. 1, it will be appreciated that the optical set-up is not limited to the arrangement shown. In particular, the illumination optical path Pᵢ and the detection optical path P_{d} may not be orthogonal at the beam splitter 70. In an alternative arrangement (not shown), the light source 10 may be arranged to emit the light beam directly towards the aperture, thus not requiring a beam splitter 70.

It is beneficial for the device 100 to remain substantially stationary and/or stable with respect to the eye 200 during an OMT measurement. Any unwanted movements of the patient's head 300 relative to the device 100 and/or movements of the device 100 by the operator relative to the patient's head 300 during a measurement acquisition time may have a negative influence on the accuracy, reproducibility and reliability of the OMT measurement (e.g. since the patient's eye sockets participate in the movement of the head 300).

In a fully handheld device 100, the potential problem of relative movements between the eye 200 and the device 100 is exacerbated, since the operator's hand 400 is inherently less stable than, for example, a fixed measurement apparatus. For example, an operator's hands 400 may shake while holding the device 100, and the outstretched arm has a natural tremor (typically in the frequency range 7 Hz to 12 Hz), both of which may contribute to noise in the OMT measurement.

To support and/or stabilise the device 100 during an OMT measurement, the device 100 may comprise one or more support portions 140 or mounts 140, as shown in the example embodiment of FIG. 2. The or each support portion 140 may be configured and intended, in use, to be placed in contact with one or more locations on a patient's head 300 or face. In use, the or each support portion 140 serves to support and/or stabilise the device 100 against the patient's head 300. The or each support portion 140 may, in use, also help to maintain a predetermined position of the device 100 (particular, the light beam exiting the aperture 40 of the device 100) relative to the target area of the eye 200.

As such, the or each support portion 140 allows the device 100 to be at least partially supported by the patient's head 300 during a measurement. In this way, in use, the device 100 may be able to follow any movements of the patient's head 300, and any vibrations of the operator's hand or arm may be substantially transferred to the patient's head 300 and/or dampened. This may effectively cancel the majority of unwanted relative movements between the device 100 and the eye 200.

The or each support portion 140 may be in the form of a projection that extends from the housing 120. The distal end 140d of the or each support portion 140 may provide an interface portion 140d configured to be placed in firm contact with the patient's head. In the example shown in FIG. 2, the support portion 140 extends away from a lower side 1221 of the housing 120, such that, in use, the operator may place the support portion 140 in contact with the patient's skin around the eye socket and/or cheek beneath the eye 200 to stabilise/support the device 100. Alternatively, the or each support portion 140 may extend from a different side of the housing 120. For example, alternatively or additionally, a support portion 140 may extend away from an upper side 122u of the housing 120, such that, in use, the operator may place the support portion 140 in contact with the patient's skin around the eye socket and/or forehead above the eye 200 to stabilise the device 100. Alternatively or additionally, a support portion 140 may comprise a substantially bridge shaped interface portion 140d configured, in use, to be in contact with and supported on the bridge of the patient's nose.

The support portion 140 and/or the interface portion 140d may be shaped to conform to the one or more locations of the patient's head 300. The interface portion 140 may be or comprise a pad or interface layer for placing in contact with the one or more locations of the patient's head 300. The support portion(s) and the interface portion 140d may be formed from a substantially hard incompressible material, such as a hard plastics material. The interface portion may be ergonomically shaped to conform to one or more locations on a patient's head 300 (e.g. to fit around the patient's eye socket or bridge of the nose). Although only one support portion 140 is shown in FIG. 2, the device 100 may comprise further support portions 140 (not shown).

In an embodiment, the support portion 140 is attachable to the housing in a fixed position and/or orientation.

In the embodiment shown in FIG. 2 the device 100 comprises a handle 160 for holding and/or gripping by an operator to manipulate the device 100. Optionally or preferably, the handle 160 may comprise an ergonomic grip. The handle 160 may be integrally formed with the housing 120, or attachable to the housing 120. In the embodiment shown, the handle 160 is attached to the housing 120 in a fixed position and/or orientation. However, in another embodiment (not shown) the handle may be pivotably mounted to the housing 120 such that its position and/or orientation may be adjusted. In use, the combination of the handle 160 and the one or more support portions 140 may increase the stability of the device 100.

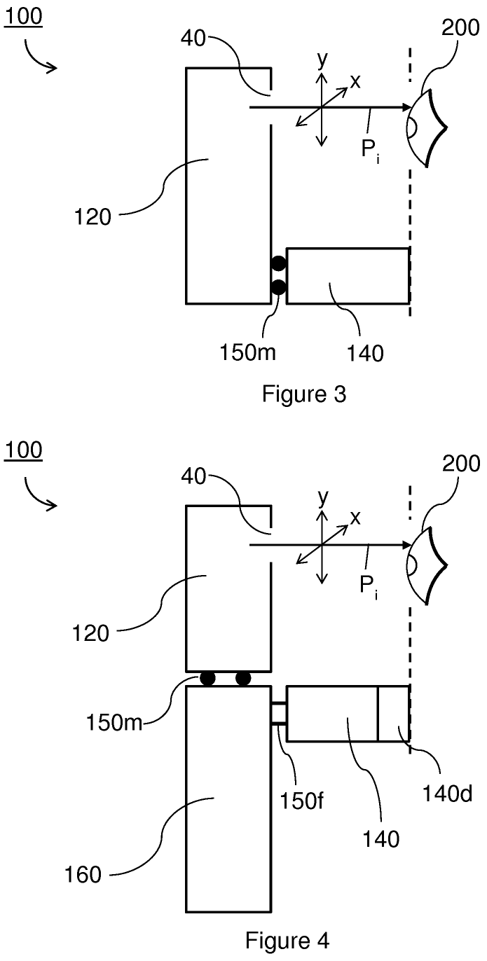

FIG. 3 shows a schematic diagram of an adjustable device 100 where the housing 120 is adjustable or moveable with respect to the support portion 140 (or vice versa). In use, the support portion 140 is placed in contact with one or more locations on the patient's head or face, as indicated by the dashed line. In this example, the supporting portion 140 may be pivotably and/or slidably mountable to the housing 120 at a movable coupling 150m, indicated by the solid circles. The orientation, angle and/or position of the housing 120 relative to the support portion 140 may be adjustable. The coupling 150m may be configured to allow the housing to move in one or more directions relative to the support portion 140, as indicated by the x and y arrows. The directions may be linear (e.g. X, Y and/or Z), or non-linear (e.g. rotation). The position of the housing 140 may be manually adjustable and/or moveable. Alternatively or additionally, adjustment and/or movement of the housing 120 may be electrically controlled, e.g. the device may be configured to adjust and/or move the support portion(s) relative to the housing (or vice versa) in response to an operator input. A handle 160 (not shown) may be moveably or fixedly mounted to the housing 120 e.g. at the bottom of the housing 120 as shown in FIG. 3.

In embodiments where the housing 120 is configured to move relative to the support portion 140 (e.g. via the moveable coupling 150m), the handle 160 may move with the housing 120.

In an alternative embodiment, the handle 160 may be integrally formed with the support portion 140, or attachable to the support portion 140. As such, in embodiments where the housing 120 is configured to move relative to the support portion 140 (e.g. via the moveable coupling 150m), the handle 160 may not move with the housing 120.

FIG. 4 shows an embodiment of an adjustable device 100 having a housing 120, support portion 140, and a handle 160, where the handle 160 is attached to the support portion 140 in a fixed position and/or orientation (at a fixed coupling 150f), and the housing 120 is adjustable and/or moveable with respect to the handle 160 and support portion 140. In this example, the housing 120 is coupled to the handle 160 via a moveable coupling 150m.

In an alternative embodiment of an adjustable device 100, both the housing 120 and the support portion 140 are adjustable and/or movable with respect to the handle 160 (not shown). For example, both the housing 120 and the support portion 140 may be coupled to the handle 160 via a moveable coupling 150.

In an embodiment, the moveable coupling 150m is a manual and/or electrically controlled multi-axis translation stage (e.g. using piezo actuators, magnetic drives, linear motors, encoders, or motors, or other translations stage technologies known in the art). The translation stage may be a 2-axis stage configured to move the housing in the X and Y directions (e.g. in the vertical plane). Optionally, the translation stage may be a 3-axis stage configured to also move the housing in the Z direction (e.g. in the horizontal plane). The coupling 150m may further permit rotation and/or tilt of the housing 120 relative to the support portion 140. Movement of the housing 120 relative to the support portion 140 allows the device 100 (in particular the light beam Pᵢ exiting the port 40) to be aligned to the target area of the eye 200 during setup of the measurement. It may also allow the device 100 to be adjusted to fit or measure OMT in patients with different sized heads.

In use, the or each support portion 140 may also provide a pivot point against the patient's head 300 about which an operator may pivot the device 100 and adjust its position, e.g. to adjust the target distance D and/or the target area (i.e. the X, Y position) of the eye 200. The interface portion 140d may be mountable to the device 100 at or near the pivot point.

The device 100 may further comprise a mirror 80 arranged to direct the light beam to the aperture 40 through the beam splitter 70, as shown in FIG. 1. Alternatively, the mirror 80 may be arranged to direct the light beam to the aperture 40 without passing through a beam splitter 70 (not shown).

The mirror 80 may be mounted in the housing 120 in a fixed position/orientation. Alternatively, the mirror 80 may be adjustable and/or moveable with respect to the housing 120, to adjust the position of the light beam on the eye 200. The movable mirror 80 may be manually operated, or electrically controlled (e.g. comprising motorised mirror mounts, piezo actuators, servos or any other moveable mirror technology). Where the mirror 80 is electrically controlled, the device 100 may be configured to move the mirror 80 in response to an operator input. The moveable mirror 80 may provide for manual or automatic alignment of the light beam to the target area of the eye, e.g. during set up. In an auto-align function, e.g. once initiated by an operator, the device 100 may further be configured to move the mirror 80 in response to a detector output signal to locate the target area whilst maintaining the scattered light substantially within the field of view of the detector 20 (i.e. to maintain an adequate detector signal for the OMT measurement). In this way, the detector output signal may be used as feedback when aligning the device.

The device 100 may be configured to emit a substantially collimated light beam for illuminating the target area of the eye 200. For example, the light source 10 may be configured to emit a collimated light beam. Alternatively or additionally, the device 100 may comprise one or more collimating optical elements (e.g. lenses) to collimate the light beam emitted from the light source 10 (not shown).

The wavelength range of interest is the wavelength (or wavelength range) of light emitted from the light source 10. The wavelength emitted by the light source 10 may be in the visible to near infrared (near-IR) range. In an embodiment, the wavelength emitted by the light source 10 may be in the range substantially 500 nm to 850 nm.

A light beam that is visible to the human the eye may be preferable for device alignment purposes. For example, such a light beam may provide a visible reference or aiming point for the operator to see and use to align the device 100, e.g. so that the light beam illuminates the target area of the eye 200. At the same time, it may also be preferable for the light beam to be only partially visible or weakly visible to the human eye, so that the patient who's eye 200 is being illuminated is not overly aware of the light beam illuminating the eye 200. It will be appreciated that the visibility of the light beam may be adjusted by the choice of wavelength and/or optical power (e.g. longer wavelengths and/or lower power light beams are less visible to the human eye).

The device 100 may comprise two light sources, each emitting light at a different wavelength. For example, one may emit light in the visible wavelength range for alignment purposes, and the other may emit light in the near-infrared wavelength range for measurement purposes. Both may emit light beams that are substantially coincident at the target area of the eye.

In an embodiment, the light source 10 emits substantially coherent and monochromatic light. For example, the light source may be or comprise a laser, such as a diode laser. Further, the light source 10 may be a low power laser diode. Alternatively, the light source may or comprise a light emitting diode. The power output of the light source may be less than substantially 3 mW, 2 mW, 1 mW, 0.5 mW, or 0.2 mW.

It will be appreciated that the optical power of the light beam exiting the aperture 40 for illuminating the eye 200 must be restricted to an eye safe level. The limit for safe exposure of eyes to light (in particular laser radiation) is dependent on the wavelength and the duration of exposure. For example, for direct viewing of a 632.8 nm wavelength laser beam with 10 s exposure time to a retina of an eye with a 7 mm pupil diameter, the maximum permissible exposure is found to be about 380 μW. In the present invention, in normal use, the target area of the eye 200 is the eye sclera (not the pupil).

The power of the light beam may be limited by the power output of the light source 10. Additionally or alternatively, the power of the light beam may be reduced from the output power of the light source 10 to a suitable eye safe level by the presence of the beam splitter 70 and/or one or more power attenuating elements, such as a neutral density filter (not shown). The device 100 may further comprise a shutter (not shown) to selectively block the illumination path Pᵢ and/or the aperture 40, e.g. when a measurement is not being taken.

The OMT measurement is based on optical interferometry. In use, the light beam is directed along the illumination optical path Pᵢ to illuminate the target area of the eye 200, and scattered light from the interaction of the light beam with the surface of the target area of the eye 200 travels along the detection optical path to the detector 20. Where the surface of the target area is optically rough (i.e. having roughness on the scale of the wavelength of light illuminating the eye 200), the scattered light from the target area of the eye 200 forms an interference pattern known as a speckle pattern, that is specific to the surface of the illuminated area. The interference pattern can be imaged by the detector 20, e.g. in the Fourier plane of the focusing lens 30. Any movement of the eye 200 relative to the light beam illuminating the target area of the eye 200 alters the surface of the illuminated target area, which in turn alters or displaces the speckle pattern. The measurement of OMT is based on analysing the changes in the speckle pattern over a predetermined measurement time interval (acquisition time). For this purpose, the detector 20 may be a one-dimensional or two-dimensional imaging sensor such as a high speed digital camera with a frame rate faster than (at least twice) the typical OMT frequencies (i.e. up to 150 Hz) to capture the changes in the speckle pattern. As such, the detector 20 may have a frame rate of at least 300 fps. In an embodiment, the frame rate is 500 fps.

A software algorithm may be used to process the images captured by the detector 20 and extract the features of interest, namely the dominant microtremor frequency and/or the mean microtremor amplitude. Such image processing algorithms have previously been described by E. Kenny et al. in Journal of Biomedical Optics 18(1), 016010 (2013) and involve cross-correlating the digital images captured in the measurement time interval to determine a linear pixel displacement pₓ between speckle images. In the Fourier plane technique, the linear pixel displacement can be mapped to the (out of plane) angular rotation AO of the eye 200 using the simple equation Δθ=pₓ/2f, where f is the focal length of the focusing lens 30.

A typical measurement (acquisition) time interval may be 3 s, in which the detector 20 may capture up to 1500 image frames (e.g. captured at a frame rate of 500 fps). In an embodiment, the measurement time may be in the range of substantially 2 s to 10 s, or 2 s to 8 s, 2 s to 6 s, or 3 s to 6 s. Images starting from the second captured image frame are cross-correlated with the previous image frame. Alternatively, the first captured image frame can be cross-correlated with each subsequently captured image frame. The cross-correlation results in a cross-correlation peak representing the relative displacement between speckle patterns in the cross-correlated image frames, at a pixel resolution. Optionally, to increase the accuracy of the result, namely the resolution of the peak, an interpolating sub-pixel algorithm based on curve fitting may be implemented, such as that described by Hung et al., in the Journal of the Brazilian Society of Mechanical Sciences and Engineering, p 215-221, 25(3) (2003). The sub-pixel algorithm effectively increases the number of pixels, thereby increasing the displacement measurement resolution of the cross-correlation peak. In some cases, the measurement resolution may be increased by almost a factor of 10. Optionally, any ambiguous cross-correlation results, which may arise from noise in the captured images or sudden movements of the eye 200 or patient's head 300, may be rejected. The eye movement data or OMT signal is constructed from the change in the cross-correlation peak location over time. The change in the cross-correlation peak location over time is used to calculate the linear pixel displacement pₓ of the speckle images over time pₓ(t). Because the linear pixel displacement pₓ in the speckle pattern maps to the angular rotation/displacement of the eye 200, the angular displacement of the eye 200 over time (i.e. eye movement data) can be calculated. The mean microtremor amplitude (e.g. peak-to-peak) can be readily determined from the eye movement data. The frequency of the microtremor can also be readily determined from the eye movement data, e.g. by counting peaks over time and/or by known Fourier analysis techniques (e.g. by performing a Fourier transform). Optionally, any noise in the eye movement data at frequencies outside the frequency range of interest (i.e. the typical/expected range of OMT frequencies) may be removed by suitable band pass filtering before analysis of the microtremor amplitude and/or frequency.

The algorithm may also implement active vibration cancellation in the processing of captured image frames.

The above measurement analysis may be performed by a processor, based on the detector output signal. The processor may be configured to complete the microtremor measurement analysis in less than 1 minute, thus providing rapid measurement feedback to the operator. The device 100 may comprise the processor, allowing the OMT measurement and analysis to be performed by the device 100. Alternatively, the device 100 may be connectable (e.g. during and/or after a measurement) to a separate computing device having a processor (e.g. a laptop or PC) for handling the recording of captured images and/or image processing. For example, the processor may be configured to execute the software algorithm and perform the image processing steps, as described above.

The software algorithm may incorporate a clinical decision algorithm based on standardised norms of the OMT properties. For example, the decision algorithm may provide a test result, e.g. to indicate an assessment of various neurological conditions, such as concussion. The test results may be determined by comparing the OMT frequency and/or amplitude to a predetermined threshold value. The test result may be presentable as a score and/or a traffic light indicators corresponding to a particular clinical indication (e.g. not concussed, mildly concussed, concussed, etc.). For example, the device 100 may be used in pitch-side concussion testing for various sports, and the outcome of the test may be: green—indicating “safe to return to play”; or red—indicating “concussion detected”.

The device 100 may further comprise one or more output elements 185 configured to indicate information to the operator. The one or more output elements 185 may further be configured to indicate (e.g. visually and/or audibly) measurement instructions, results, feedback, and/or guidance to an operator. For example, the device 100 may comprise a display screen 185, as shown in FIG. 2, and/or one or more light emitting devices. The display screen 185 may be positioned on the housing 120 and be configured to indicate various types of information to the operator, such as a device 100 status, measurement status, and/or a measurement result. The display screen 185 may further configured to indicate a time date stamps results and/or operator instructions.

The device 100 may further comprise a communications unit configured to facilitate data communication with a computing device (not shown). The communications unit may enable data transfer between the device 100 and a computing device, database, server, or cloud server (e.g. via GSM, LORA, NBIOT or other connectivity), e.g. for data recording/storing and/or for analysis. The communications unit may be in data communication with the processor for receiving the OMT measurement data. In use, the communications unit may be in wired or wireless (e.g. Bluetooth or WiFi) communication with the computing device. Alternatively, the device 100 and/or the communications unit may comprise a memory in communication with processor for storing the OMT measurement data, e.g. for later retrieval and/or transmission to the computing device, database, server, or cloud server.

Various noise and interference sources may be manifest in the image frames captured by the detector 20. The information content of individual image frames and/or the stream of image frames may be degraded over the course of a measurement time interval by, for example, sudden relative movements of the eye and device 100, biospeckle effects, changes in lighting conditions, drift in alignment of the device 100, etc. In particular, relative device motion arising from the operator's hand represents an inherent source of additional noise in hand held device configurations. Such noise sources may negatively impact on the accuracy/quality/reliability of eye displacement information that can be recovered by cross-correlating image frames (as described above). It is therefore important to consider and minimise the impact of such noise sources on the OMT measurement accuracy/quality/reliability, particularly in hand-held device configurations.

According to an embodiment, the processor is configured to determine various quality metrics to indicate individual frame quality and/or the quality of frame-to-frame cross-correlation that can indicate accuracy/reliability of the OMT measurement. This information can then be provided to the operator as feedback (e.g. via the one or more output elements 185) and/or stored/associated with the OMT measurement (e.g. in the memory) for evaluating the quality of the OMT measurement in real-time and/or at a later time.

The processor may be configured to determine one or more image frame quality metrics of individual image frames for some or all of the captured image frames based on the respective image frame data. The image frame quality metrics may include but are not limited to the total integrated signal across the image, and/or shape (e.g. height and width) of auto correlation peak, speckle parameters (e.g. size, intensity, contrast), etc. These may be determined during or after image capture.

In addition, the processor may be configured to determine one or more inter-frame correlation quality metrics of cross-correlated images based on the cross-correlation data to assess the uncertainty in the estimate of the relative displacement of frames from the cross-correlation. The result of the cross-correlation of two successive image frames is the cross-correlation peak superimposed on top of a background/floor signal which represents the background noise. The more correlation between the two images, the greater height the peak has above the background noise level. Conversely, the more displacement between the two image frames the lower the correlation. As such, excessive operator induced device movement may manifest as poor inter-frame correlation and low peak height. The lower the correlation between images means there is greater chance that the peak location does not accurately map to the spatial displacement between the images (and thus the angular eye movement). The inter-frame correlation quality metrics can be based on a variety of quantitative parameters derived from the cross-correlation of image frames, including but not limited to the ratio of the cross-correlation peak height to mean floor/background amplitude, and/or the ratio of the correlation peak height to the variance of floor/background signal, and/or other quantitative signal-to-noise metrics known in the art. The specific relationship between the inter-frame correlation quality metric(s) and the uncertainty in the estimate of frame-to-frame displacements may be established though empirical or deterministic means.

In some examples, the determined image frame quality metric(s) and/or inter-frame correlation quality metric(s) are compared to predefined threshold values to identify poor quality image frames that are likely to carry little to no valuable information for the purpose of OMT measurements and/or frame-to-frame correlations that are associated with an unacceptable level of inaccuracy in frame-to-frame displacement estimations, respectively. It will be appreciated that the threshold values will be dependent on the specific metric. Less uncertainty in the cross-correlation output is associated with a better overall accuracy/reliability of the eye movement data.

The threshold values may be an upper and/or lower bound. Image frames with one or more image frame quality metrics, or pairs of image frames with one or more inter-frame correlation quality metrics, falling outside the predetermined threshold values are likely to carry little to no valuable information for the purpose of OMT measurement. The processor may be configured to reject these “poor quality” image frames prior to further downstream processing to prevent introducing unreliable measurement points. This may also reduce the downstream processor load. The remaining “good quality” image frames that are of sufficient quality and are sufficiently correlated can then be used for eye movement data construction and OMT parameter extraction. For example, the processor may be configured to reject image frames with one or more image frame quality metrics falling outside the predetermined threshold values prior to the cross-correlation stage, and/or reject pairs of image frames with one or more inter-frame correlation quality metrics falling outside the predetermined threshold values prior to constructing the eye movement data. Where a pair of image frames has one or more inter-frame correlation quality metrics falling outside the predetermined threshold values, it may be the case that only one of the image frames is of poor quality, In this case, the individual image frame quality metrics of each of the pair may be used to identify the poor quality image frame to be rejected.

The above described quality metrics can be used to provide feedback information to the operator for the purpose of adjusting alignment and/or stability of the device 100 in real time. This may be provided by the one or more output elements 185, such as a display screen. In particular, this feedback feature may advantageously be used to help achieve acceptable operational requirements in a hand-held device configuration. At the end of the measurement time interval, individual frame and/or frame-to-frame quality metrics may be combined to give the operator feedback information indicative of the overall quality of the OMT measurement. The feedback information provided may be based on average quality metric values determined for each measurement time interval. The feedback information provided may be the quantitative (averaged) quality metric values themselves or other qualitative information derived from the quantitative quality metric values such as simplified numerical and/or textual quality scales (e.g. 1 to 10, poor to good, and/or traffic light scales).

For example, a characteristic set of values (or ranges thereof) of image frame quality and/or image frame correlation quality metrics can be used to identify whether the captured speckle images are formed by the reflection of light from the target eye area as intended to be used for OMT measurement (such as the sclera), or an erroneous area (e.g. iris or eye lid). In addition, continuous stability feedback can be provided to the user while the measurement takes place.

Further, where the mirror 80 is moveable to provide automatic alignment of the light beam on the target area of the eye 200, a feedback loop can be implemented using signals derived from the above described quality metrics to automatically optimise alignment. For example, the mirror 80 may be adjusted (to adjust the position of the light beam on the eye 200) based on the signals derived from the above described quality metrics to optimise the quality metrics. These metrics can be also used to derive input signals to an active stabilisation mechanism to counteract unwanted motion artefacts by maintaining the stability of the device or its specific components. For example, the mirror 80 may be dynamically adjusted during measurements based on feedback derived from the above described quality metrics to maintain steady projection of the light beam.

Sudden movements of the device 100 relative to the eye 200 or vice versa may manifest as high velocity jumps and appear as “steps” in the eye movement data. The velocity of device-to-eye relative movements can be in excess of 10-20 times the typical velocity of OMT. Excessive inter-frame velocity may be incompatible with downstream signal filtering and produce ringing effects which can mask the small amplitude OMT signal in the eye movement data. Accordingly, the processor may be configured to identify and reject image frames having inter-frame velocities above a predefined threshold prior to signal filtering. The inter-frame velocity can be determined from the change in the cross-correlation peak position between successive image frames. The threshold velocity or the limit of tolerable velocity with an acceptable impulse response can be established deterministically or empirically.

Additionally or alternatively, the device 100 may be configured to actively track motion of the device 100 relative to the subject's head and remove or suppress any corresponding artefacts in the eye movement data arising from such device-head movements (as may be present in hand-held device configurations). Such device-head motion arising from the operator's hand may be quasi periodic and may result in rotational motion of the device relative to the head and eye, and thus be manifest in the speckle images. In particular, the device 100 may be configured to detect motion of a reference target relative to the device 100, which can be used to remove/suppress the unwanted device-head motion component in the net motion captured from the eye in the eye movement data, e.g. by subtraction. The remaining motion is then ‘clean’ eye movement data. The reference target is configured to follow the motion of subject's head relative to the device 100 directly or indirectly. The reference target can be a natural anatomical feature of the subject's head such as the forehead that can facilitate a reliable reference of relative head movement, or the reference target can be an artificially introduced object that can facilitate tracking of the head motion relative to the device 100. In the latter case, the reference target may be or comprise an object coupled to or worn on the head of the subject. As such, the reference target can be separate from or part of the device 100 but is coupled (e.g. optically) in the way that the detector 20 in the device 100 can track the head motion. The device 100 may be configured to measure relative head-to-device motion through a variety of means, for example optical tracking based on laser speckle interferometry, laser interferometry or other means, or by electronic sensing using e.g. capacitive sensing.

In one example, the reference target may be or comprise a surface from which a laser beam is reflected or scattered from, according to the motion of subject's head. The surface is appropriate to the optical measurement technique employed and may be a mirror, an optically rough surface or a combination of optical elements. Measurement of the reference surface motion may be achieved through optical speckle metrology as described above or optical interferometry as is known in the art. The device may be configured to provide two independent and simultaneous motion measurements, with one incident light beam measuring eye movement as described above and another incident light beam measuring displacement of the reference surface relative to the device 100. The measured movement of the reference surface can then be subtracted from the net motion captured from the eye in the eye movement data.

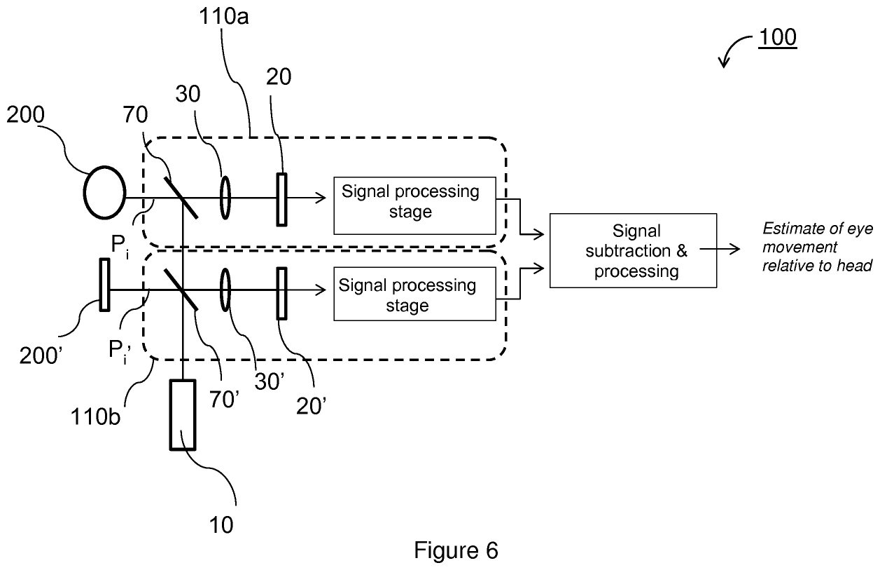

FIG. 6 shows an example configuration of a device 100 comprising two measurement sub systems 110a, 110b, each configured as previously described to be sensitive to angular movements of the respective target. In this example, a single light source 10 is used to provide two incident light beams Pᵢ and Pᵢ′, one of the target 200 (Pᵢ) and one for the reference target 200′(P₁′). However, it will be appreciated that separate light sources 10 could be used for each sub-system 110a, 110b. Each sub-system 110a, 110b is in a speckle measurement configuration with the detector 20, 20′ in the Fourier plane of the lens 30, 30′. One subsystem 110b generates a signal estimating the angular movement of the device 200 relative to the head, The other subsystem 110a generates a signal estimating the angular movement of the eye 200 relative to the device 200. Any small unwanted linear displacement may produce some decorrelation between image frames, but no systematic shift in the captures speckle patterns. The signals are subtracted or otherwise combined to provide an estimate of the angular movement of the eye relative to the head.

In another example, the device 100 may be configured optically to be sensitive primarily to relative eye/head movements. In this example, the reference surface or optical element can be included the optical path of the laser beam as it passes from device 100 to the eye 200 and back to the detector 20. The optical configuration is designed so that any relative device-head movement produces compensatory motion in the speckle image falling on the detector. For example. in the Fourier plane speckle method used to measure eye movements described above, any optical configuration that provides an angular correction to the incident or scattered laser light on or from the eye 200 in response to relative angular movement of the device 100 and the reference target is a candidate for reducing the impact of relative head/device movements on OMT measurement.

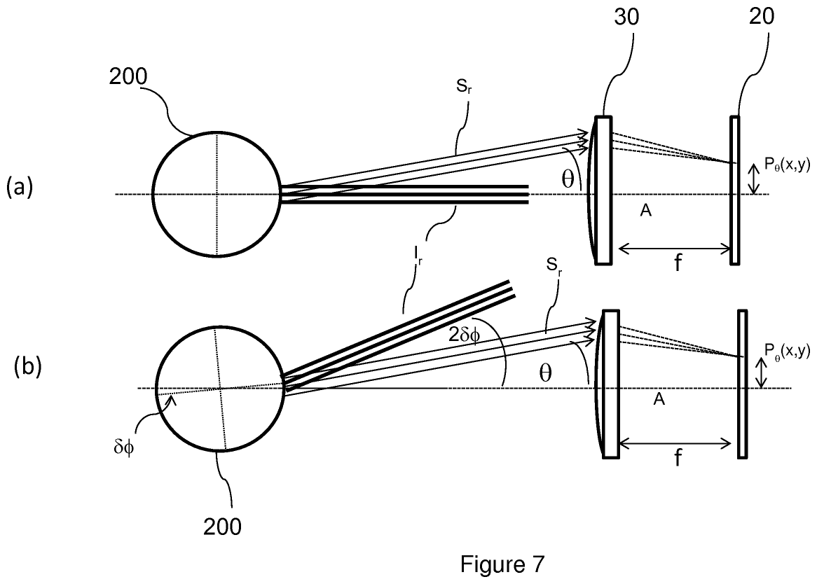

FIGS. 7a and 7b illustrate the above approach. In the Fourier configuration, rays scattered Sᵣ from the surface of the target 200 at some angle θ relative to a fixed axis A of the device 300 are brought to convergence at a point P_{θ}(x,y) on the detector 20, as shown in FIG. 7a. The overall speckle pattern is formed by the lens 30 collecting and converging rays scattered Sᵣ from the illuminated area in multiple angular directions. Any small unwanted linear displacement of the light beam relative to the target 200 may produce some decorrelation between image frames, but no systematic shift in the speckle pattern. Any small unwanted angular movement δϕ of the device 100 relative to the head (see FIG. 7b) introduces some unwanted movement in the overall speckle pattern. However, if the laser beam illuminating the target 200 is given a compensatory angular adjustment 2δϕ relative to the device 200 (which can be achieved e.g. through the adjustable mirror 80, not shown), then the point of convergence P_{θ}(x,y) on the detector 20 of light scattered from the angle θ remains unchanged, as shown in FIG. 7b.

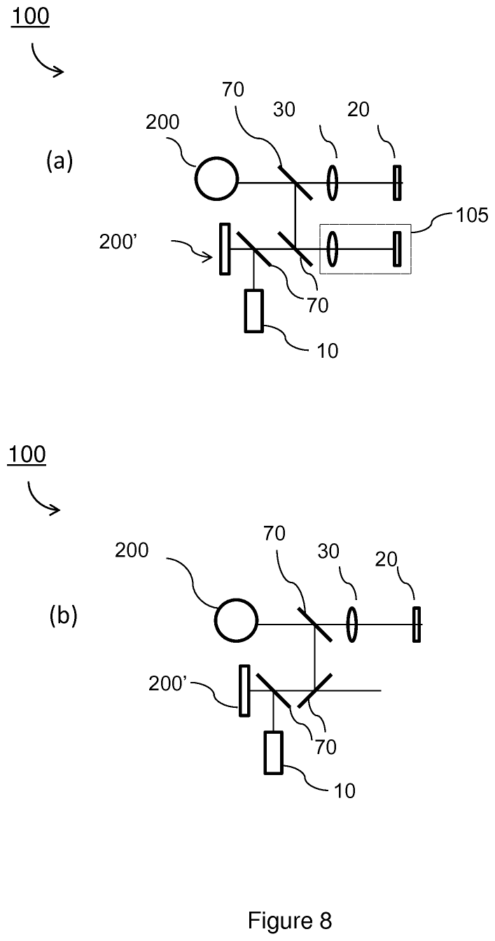

The required compensatory angular adjustment 2δϕ to the incident light beam can be achieved by first directing (e.g. via a beam splitter 70) the laser beam to a reference target 200′ on the head, such as a mirror. The light beam is then returned to optical elements in the device 100 with any angular adjustment introduced by misalignment of the device 100 to the reference target 200′. The configuration is such as to introduce the appropriate angular correction 24 in the light beam directed to the target 200. Essentially, any configuration that sends the light beam to the target 200 parallel but opposite to the direction in which the light beam is reflected from a plane mirror acting as a reference target 200′ will provide the required angular correction. Two example configurations are shown in FIGS. 8a and 8b. FIG. 8a compensates for small angular movements of the device 100 relative to the reference target 200′ (mirror) in two axes and FIG. 8b compensates for small angular movements of the device 100 relative to the reference target 200′ (mirror) in just one axis. In FIG. 8a, the light beam from the device 100 is reflected off a reference mirror 200′ back to a cat's eye or other retroreflector element 105 in the device 100. The outgoing beam from the retroreflector 105 is parallel to the incoming beam (i.e. the light beam reflected off the mirror 200′), meeting the requirement described for correction of small relative angular device/head movements.

A series of optical elements (such as beam splitters 70) then directs this light beam to the eye 200, as shown. In FIG. 8b, the light beam is sent to the eye 200 from the reference mirror 200′ using a simple combination of beam splitters 70. In this case, the light beam reflected from the reference 200′ and the light beam sent to the eye 200 will only be parallel in one plane and only for small angular movements.

Alternative configurations can be envisaged where the angular correction is introduced in the light scattered from the eye 200 as it returns to the detector 20 via a reference element, rather than by correcting the direction of the illuminating light beam.

The device 100 may comprise one or more operator inputs 180 for operating the device 100. The one or more operator input(s) 180 may be or comprise one or more buttons and/or switches, for example, to turn the device 100 on and off, configure an OMT measurement, and/or start/stop an OMT measurement. The handle 160 may comprise at least one of the one or more operator inputs 180, e.g. to start a measurement. The device 100 may be operable to send or transmit data to another computing device or server/database via the communication unit data in response to an operator interacting with one or more of the operator inputs.

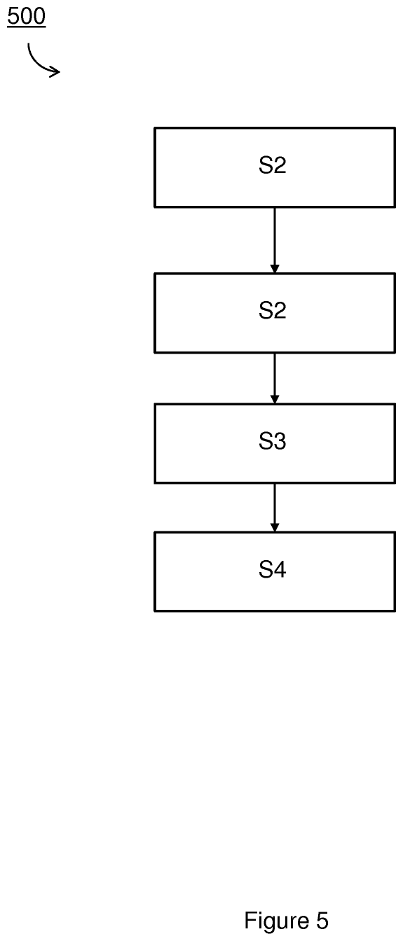

FIG. 5 shows a method 500 of measuring OMT using the device 100. In step S1, the device 100 is positioned or placed in contact with a patient's head or face to support and/or stabilise the device. This may comprise positioning the device 100 such that support portion(s) 140 of the device 100 is placed in contact with one or more locations on a patient's head or face. In step S2, the device 100 is aligned to a target area of the patient's eye 200. This may comprise aligning the housing 120 to within a predetermined volume with respect to the eye. In step S3, the target area of the eye 200 is illuminated with a light beam emitted from the device 100 and scattered light from the interaction of the light beam with the target area of the eye 200 is detected at a detector 20 of the device 100. In step S4, one or more microtremor properties are determined or measuring based on an output signal of the detector 20. The one or more microtremor properties may be or comprise one or more amplitude and/or frequency components of the output signal. The method 500 may further comprise providing and/or displaying at least one of the one or more microtremor properties to an operator.

Step S4 may comprise recording an output signal of the detector for a period of time, and processing the recorded output signal to determine one or more microtremor properties. Step S4 may further comprise capturing a series of images of the scattered light at the detector, and processing the captured image frames to determine one or more microtremor properties. Capturing the series of image may comprise capturing at a frame rate of at least twice the highest OMT frequency of the OMT frequency band of interest. Processing the captured images may comprise cross-correlating the images to determine an OMT trace representing movement of the eye versus time. Processing the captured images may further comprise filtering the OMT trace to remove or attenuate frequencies outside of the OMT frequency band of interest (e.g. to remove signals associate with unwanted eye motions such as microsaccades).

Processing the captured images may comprise (prior to cross-correlation): determining, for one or more image frames of the series of images, one or more image frame quality metrics based on the respective image frame data; comparing, for each of the one or more image frames, the one or more image frame quality metrics to one or more predefined threshold values; and rejecting or deleting the images frames with one or more image frame quality metrics falling outside the one or more predefined threshold values. Cross-correlating the images may comprise cross-correlating adjacent pairs of image frames in the series of image frames. Cross-correlation of a pair of adjacent image frames may produce cross-correlation data for the respective pair of cross-correlated images. The OMT trace may be determined from the location of the cross-correlation peak in the cross-correlation data of each pair of cross-correlated images. Processing the captured images may comprise (after cross-correlation): determining, for one or more of the adjacent pairs of images frames, one or more inter-frame correlation quality metrics based on the cross-correlation data, comparing the one or more inter-frame correlation quality metrics to one or more predefined threshold values; and rejecting or deleting the (pairs of) images frames with one or more inter-frame correlation quality metrics falling outside the one or more predefined threshold values. Processing the captured images may comprise: determining an inter-frame velocity based on the change in location of the cross-correlation of successive pairs of cross-correlated images; comparing the inter-frame velocity to a predefined threshold value; and rejecting or deleting the image frames with an inter-frame velocity exceeding the threshold value.

Step S2 may further comprise aligning the light beam with the target area of the eye, and optionally or preferably, adjusting and/or moving the housing and/or support portions to align the light beam with the target area of the eye. Aligning the device may further comprise adjusting and/or moving the housing relative to the support portion(s).

From reading the present disclosure, other variations and modifications will be apparent to the skilled person. Such variations and modifications may involve equivalent and other features which are already known in the art, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination.

For the sake of completeness it is also stated that the term “comprising” does not exclude other elements or steps, the term “a” or “an” does not exclude a plurality, and any reference signs in the claims shall not be construed as limiting the scope of the claims.

1. A device for measuring ocular microtremor (OMT) of a patient's eye, comprising:
a light source for illuminating a target area of the eye with a light beam;
a detector arranged to detect scattered light from the interaction of the light beam with the target area of the eye;
a focusing lens arranged to collect the scattered light for the detector; and
a port in a wall of the device through which the light beam can exit the device and/or through which the scattered light can enter the device; and
wherein the device is configured to stabilise and/or support the device on or against a patient's head.

2. The device of claim 1, wherein the lens is positioned a predefined position from the detection and/or the detector is positioned in a Fourier plane of the focusing lens.

3. The device of claim 1 or 2, further comprising one or more support portions configured, in use, to be placed in contact with one or more locations on a patient's head or face and, optionally or preferably, to conform to one or more locations on a patient's head or face.

4. The device of claim 3, wherein the one or more support portions are configured, in use, to position the port at a predetermined distance from said eye during a measurement; and optionally or preferably, within a predetermined volume of space with respect to said eye.

5. The device of claim 3 or claim 4, wherein the position of the light source, the detector and the focusing lens relative to the or each support portion adjustable.

6. The device of any of claims 3 to 5, wherein the light source, the detector and the focusing lens are arranged within a housing, and the housing is moveable relative to the or each support portion to position; and optionally or preferably, wherein the moveable housing is configured to position the port to within a predetermined volume of space with respect to said eye.

7. The device of claim 6, wherein the housing is pivotably and/or slidably coupled to the or each support portion, and optionally or preferably, wherein the device configured to move the housing in one or more directions in response to an operator input.

8. The device of any of claims 3 to 7, wherein the or each support portion comprises an interface portion at a distal end of the or each support portion; and, optionally or preferably, wherein the or each support portion and/or the interface portion is ergonomically shaped to conform to one or more locations on a patient's head or face.

9. The device of claim 8, wherein the or each support portion and/or the interface portion are formed of or comprise a substantially hard or non-deformable material.

10. The device of any preceding claim, wherein, the detector is arranged and configured, in use, to image an interference pattern in the Fourier plane of the focusing lens formed from the scattered light.

11. The device of claim 10, wherein the detector is or comprises an image sensor configured to record images of the interference pattern over a period of time; and optionally or preferably, wherein the imaging sensor has an image capture frame rate of at least 300 Hz.

12. The device of any preceding claim, configured to determine one or more ocular microtremor properties of said eye based on an output signal of the detector; and optionally or preferably, wherein the one or more ocular microtremor properties include a microtremor frequency and/or a microtremor amplitude.

13. The device of any preceding claim, comprising, or configured to be connectable to, a processor configured to perform the ocular microtremor measurement based on an output signal of the detector.

14. The device of any preceding claim, comprising one or more operator inputs to operate the device.

15. The device of any preceding claim, comprising one or more output elements configured to provide one or more output signals to an operator; and optionally or preferably, wherein the one or more output signals is or comprises a visual, audio and/or a haptic signal.

16. The device of claim 15, wherein the one or output signals are configured to indicate measurement information including one or more of: a measurement status, a measurement result, a microtremor amplitude, and a microtremor frequency; and optionally or preferably, wherein at least one of the output elements is a display screen.

17. The device of any preceding claim, wherein the device is a hand held device.

18. The device of any preceding claim, further comprising a handle portion for manipulating the device; and optionally or preferably, wherein the handle portion comprises an ergonomic grip and, optionally or preferably, wherein the handle portion comprises at least one of the operator inputs to operate the device.

19. The device of claim 18 when dependent from any of claims 6 to 17, wherein the handle portion is attachable to the or each support portion and/or the housing; and optionally or preferably, wherein the position and/or orientation of the handle portion with respect to the or each support portion and/or the housing is adjustable.

20. The device of any preceding claim, wherein the focusing lens has a focal length, and the detector is positioned at a distance substantially equal to the focal length from the lens.

21. The device of any preceding claim, comprising an illumination optical path by which the light beam reaches the port from the light source, and a detection optical path by which the scattered light reaches the detector.

22. The device of claim 21, further comprising a beam splitter arranged in the illumination optical path to direct the light beam towards the port and/or arranged in the detection optical path to direct the scattered light to the detector.

23. The device of claim 21 or 22, further comprising a mirror arranged in the illumination optical path to direct the light beam towards the port; and optionally or preferably, wherein the position and/or orientation of the mirror is adjustable.

24. The device of any of claims 13 to 23, wherein the processor is configured to:
receive an output signal from the detector, the detector output signal comprising a stream of image data comprising a series of image frames captured over a period of time at a frame rate; and
reject or delete image frames from the image data having one or more image frame quality metrics falling outside one or more predefined threshold values; and
determine an OMT signal from the remaining image data representative of eye movement over time; and, optionally or preferably,
wherein the one or more image frame quality metrics include a total integrated signal across a respective image frame, and/or a height and/or width of an auto correlation peak obtained from a respective image frame.

25. The device of any of claims 13 to 24, wherein the processor is configured to:
receive an output signal from the detector, the detector output signal comprising a stream of image data comprising a series of image frames captured over a period of time at a frame rate;
reject or delete pairs of image frames from the image data having one or more inter-frame correlation quality metrics falling outside one or more predefined threshold values; and
determine an OMT signal from the remaining image data representative of eye movement over time; and, optionally or preferably,
wherein the one or more inter-frame correlation quality metrics include one or more signal-to-noise parameters extracted from the cross-correlation of a respective pair of image frames.

26. The device of any of claims 13 to 25, wherein the processor is configured to:
receive an output signal from the detector, the detector output signal comprising a stream of image data comprising a series of image frames captured over a period of time at a frame rate;
reject or delete image frames from the image data having an inter-frame velocity exceeding a predefined threshold value; and
determine an OMT signal from the remaining image data representative of eye movement over time; and, optionally or preferably,
wherein the inter-frame velocity is based on the change in cross-correlation peak position between successive pairs of cross-correlated image frames.

27. The device of any of claims 24 to 26 when dependent from claim 15, wherein the processor is configured to provide one or more feedback signals to the one or more output elements for providing device alignment and/or stability feedback to the operator, based on the one or more image frame quality metrics and/or inter-frame correlation quality metrics.

28. The device of any preceding claim, comprising:
a first sub-system comprising the detector and the focusing lens, the first sub-system configured to provide a first signal representing angular movements or displacements of said eye relative to the device based on an output signal of the detector; and
a second sub-system comprising a second detector arranged to detect scattered light from the interaction of the light beam with a reference target at or on the patient's head and a second focusing lens arranged to collect the scattered light for the second detector, the second sub-system configured to provide a second signal representing angular movements or displacements of said head relative to the device based on an output signal of the second detector, and
wherein the device is configured to provide an OMT signal representing angular movements or displacements of said eye relative to said head based on the first and second signals.

29. A method of measuring microtremor of an eye, comprising:
providing a device as defined in any of claims 1 to 28;
supporting and/or stabilising the device on or against a patient's head;
aligning the device to a target area of the eye;
detecting scattered light from the interaction of the light beam with the target area of the eye; and
determining one or more microtremor properties of the eye based on an output signal of the detector.

30. The method of claim 29, wherein the device comprises one or more support portions, the method further comprising the steps of: placing the one or more support portions in contact with one or more locations on a patient's head or face; and adjusting the position of the light source, detector and focusing lens to align the device to the target area of the eye.
